(19) United States
(12) Patent Application Publication
(10) Pub. No.: US 2001/0051354 A1
(43) Pub. Date: Dec. 13, 2001

(54) Method for detecting microorganisms and a support which can be used in such a method

(76) Inventors: Daniel Monget, Saint Sorlin En Bugey (FR); Jean-Marc Roche, Moutiers (FR)

Correspondence Address: James C. Lydon, Suite 100, 100 Daingerfield Road, Alexandria, VA, 22314, US

(21) Appl. No.: 09/870,510

(22) Filed: Jun. 1, 2001

Related U.S. Application Data
(63) Continuation of application No. PCT/FR99/03003, filed on Dec. 3, 1999.

(30) Foreign Application Priority Data
Dec. 4, 1998 (FR) 98/15501

(51) Int. Cl. C12Q 1/22

(52) U.S. Cl. 435/31

(57) ABSTRACT

This invention concerns a method for detecting the presence in a sample, contained in a sterile receptacle, of at least one microorganism (aerobic or anaerobic), the sample being in contact with a culture medium. The invention also concerns a support which can be used in such a method.

The method consists in:

adding into the receptacle at least one sterile, inert, solid support,

incubating at a suitable temperature, and

monitoring the variation in at least one characteristic related to the presence of the microorganism(s) to be detected in said receptacle.

The invention is particularly applicable in the field of diagnosis.

### DESCRIPTION

This invention concerns a method for detecting the presence in a sample, contained in a sterile receptacle, of at least one microorganism (aerobic or anaerobic), the sample being in contact with a culture medium. The invention also concerns a support which can be used in such a method.

Document EP-A-0.124.193 describes a method for the detection of both aerobic and anaerobic microorganisms. By using a receptacle suitable for the detection of microorganisms by means of changes in pressure, detection is speeded up. On the other hand, there is no support inside the receptacle and this solution cannot be inferred from this document.

It is known that aerobic microorganisms (bacteria and yeast) adhere to and colonize most surfaces which are provided for them. Such binding enhances the exchange of nutrients and therefore encourages the growth of microorganisms. Thus, the presence of a solid phase affects numerous metabolic processes, including nitrogen fixation, alcoholic oxidation, nitrification and denitrification, etc. This particular property is widely exploited in industrial microbiology.

Thus, the prior art can be defined by document US-A-5,672,484 which relates to a receptacle for detecting obligate aerobes in a sample; the receptacle includes an interior chamber containing a culture medium, and defines a free space above the medium. This document also concerns a method of preparation for such a receptacle and a method for growing obligate aerobes. A non-toxic insert impregnated with said medium provides a free surface which at least partially projects into the free space, thereby insuring oxygenation of the culture medium, increasing the oxygen supply to microorganisms introduced in the sample, and improving their metabolic processes. This insert is chosen from the following group of materials: sponge, cotton, glass fiber beads, resin beads.

However, this apparatus and process are only applicable to the detection of obligate aerobes. Moreover, explicit mention is made of the necessity of increasing oxygen exchange between the support and the confined, free, space inside the receptacle. In order to achieve this, the insert needs to be in the form of a relatively bulky single unit which occupies 25 to 80% of the inner chamber. The objective in this document is to maximise oxygen exchange outside of the contact between the insert and the culture medium. All these characteristics could only discourage those skilled in the art from attempting to use, inside the said receptacle, a sterile, inert, solid support in anaerobic conditions, and even in aerobic conditions when the quantity of the support is adjusted to obtain a layer of material with a surface area approximately equivalent to the area of the interface between the sample and the gaseous atmosphere inside the receptacle. The results obtained by the applicant during these investigations were therefore unexpected.

This invention seeks to protect a method which is significantly more widely applicable because it can be used for all microorganisms, regardless of their mode of respiration. Moreover, the arrangement of inserts or supports within the receptacle is not subject to the same degree of restriction as that which is necessary for the prior art document.

To this effect, this invention concerns a method for detecting the presence in a sample, contained in a sterile receptacle, of at least one microorganism, whatever its mode of respiration, the sample being in contact with a culture medium, characterized in that it consists in:

adding into the receptacle at least one sterile, inert, solid support,

incubating at a suitable temperature, and

monitoring the variation in at least one characteristic related to the presence of the microorganism(s) to be detected in said receptacle.

The characteristic monitored corresponds to the variation in at least one indicator, added to the receptacle before incubation, e.g. a colored or fluorescent indicator, and/or at least one physicochemical or electrical parameter, e.g. CO₂ production, pressure, turbidity, oxidation/reduction potential and/or pH.

In all instances, the sample is either biological in nature (e.g. blood, cerebrospinal fluid, pleural fluid or urine) or non-biological (e.g. water, food products, or pharmaceutical products).

Variations in the indicator(s) are detected optically through all or part of at least one of the walls (which are transparent) of the receptacle, and/or changes in the parameter(s) are detected by means of physicochemical or electrical sensors.

Preferably, the method is applicable to anaerobic microorganisms.

This method is used in a sterility test.

This invention also concerns a sterile, inert, solid support to be used in the method defined above, which, in a second embodiment, is characterized in that it is made of natural materials, e.g.:

silica beads,

small glass beads (solid, hollow or porous),

quartz particles,

grains of sand,

vermiculite, zeolite and/or feldspar particles,

glass wool and/or rock wool,

clay beads, and

cork fragments.

According to a second embodiment, the sterile, inert, solid support used in the above-mentioned method is made of artificial materials, e.g.:

polystyrene beads,

polyethylene beads,

polypropylene beads,

clusters of small polyethylene beads, with variable pore-size and dimensions,

growth supports in the form of small beads used in tissue culture,

latex beads,

gelatin-coated beads, and

resin beads.

According to a third embodiment, the sterile, inert, solid support used in the above method consists of an element of any shape made of polyethylene. Whatever the embodiment, this support consists of beads or particles with a diameter of between 1 μm and 10 mm, and especially of between 0.1 mm and 5 mm.

The figures shown are given for explanatory purposes and are not intended to be in any way limiting. They are designed to make the invention easier to understand. In each of these figures, curve 1 corresponds to the insertion of a cork support, curve 2 to the insertion of a polyethylene support, and curve 3 to no support at all (for reference purposes).

FIG. 1 shows a kinetic growth curve for Haemophilus influenzae.

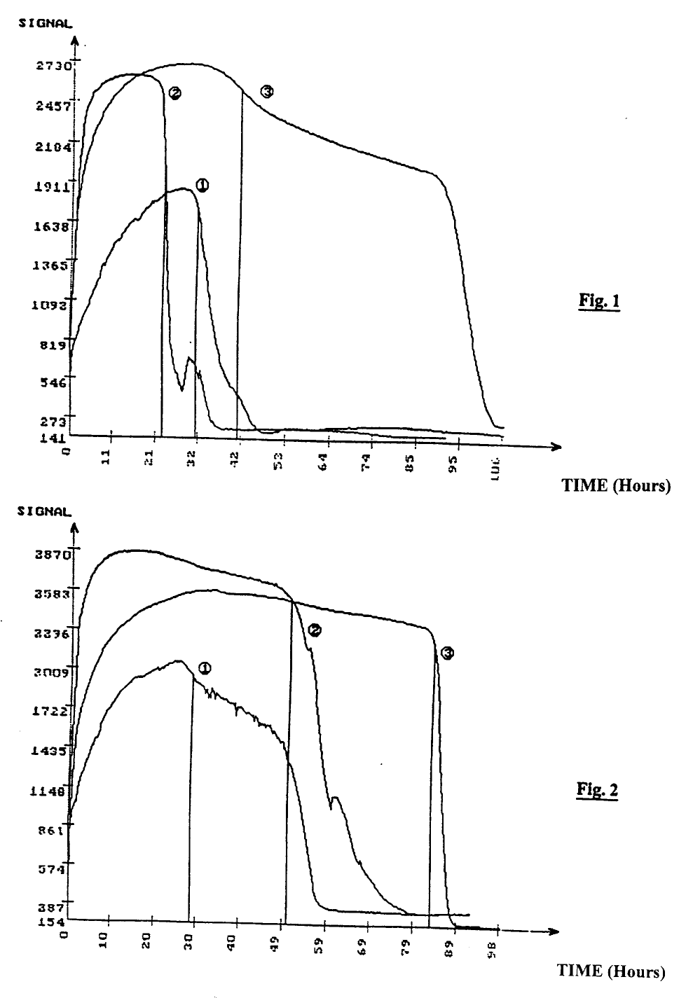

FIG. 2 shows a kinetic growth curve for Kingella denitrificans.

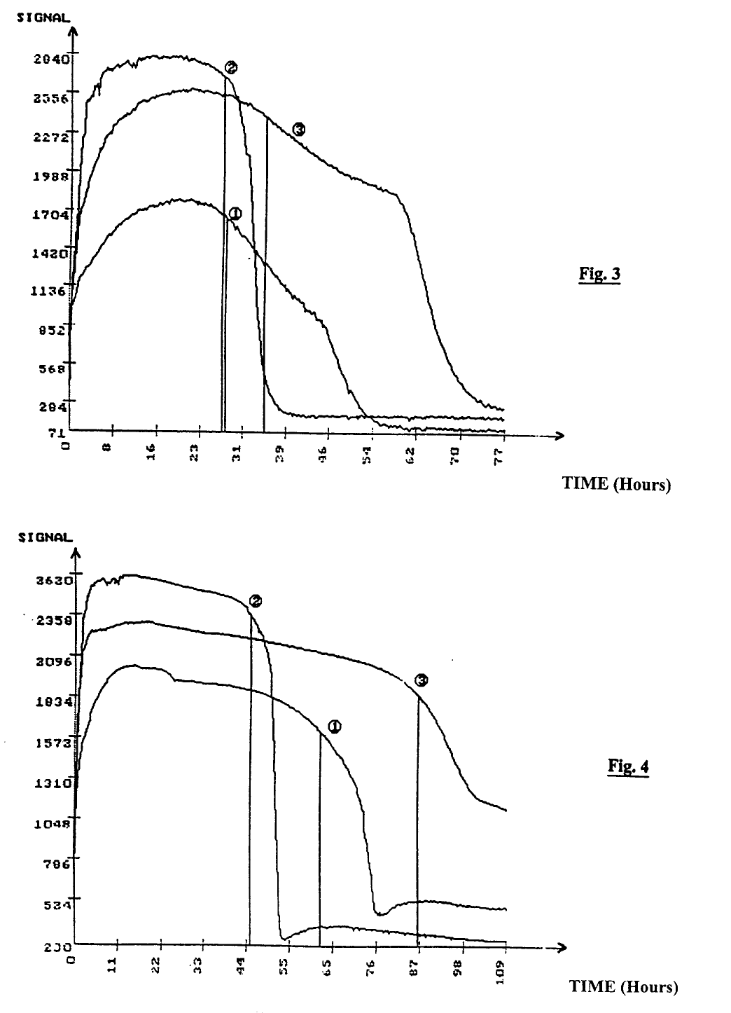

FIG. 3 shows a kinetic growth curve for Staphylococcus saprophyticus.

FIG. 4 shows a kinetic growth curve for Bacteroides fragilis.

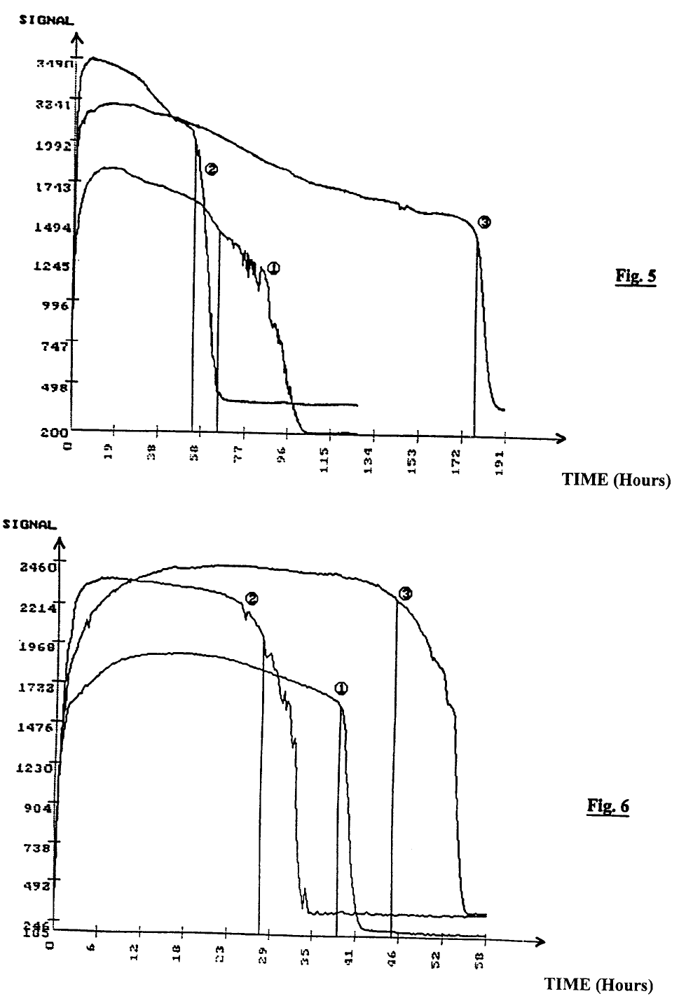

FIG. 5 shows a kinetic growth curve for Veillonella parvula.

Finally, FIG. 6 shows a kinetic growth curve for Clostridium sporogenes.

This invention concerns a method for detecting the presence in a sample, contained in a sterile receptacle, of at least one microorganism (aerobic or anaerobic), the sample being in contact with a culture medium.

In practice, detection times are reduced by the introduction of a sterile, inert, solid support into the receptacle.

The list below gives the supports which can be used to reduce the time necessary to detect microorganisms present in a sample. Of course, this list is not limiting.

In the case of the said natural supports, the following can be used:

silica beads,

small glass beads (solid, hollow or porous),

quartz particles,

grains of sand,

vermiculite, zeolite and/or feldspar particles,

glass wool and/or rock wool,

clay beads, and

cork fragments.

When it comes to the said artificial supports, the following can be used:

polystyrene beads,

polyethylene beads,

polypropylene beads,

clusters of small polyethylene beads, with variable pore-size and dimensions,

growth supports in the form of small beads for use in tissue culture available under the registered trademark CYTODEX Type 1 and Type 3,

latex beads,

gelatin-coated beads, and

diethylamino ethyl (DEAE) resin beads for anionic exchange resins or beads sold under the registered trademark SEPHADEX (CM=carboxymethylcellulose) for cationic exchange resins.

Of course, the supports added to a single receptacle can consist of a mixture of at least two of the above-mentioned artificial or natural supports, or of a mixture of at least one of the above-mentioned artificial supports together with at least one of the above-mentioned natural supports.

Implementation:

The implementation presented concerns a specific embodiment although alternatives are possible. Therefore, this implementation cannot be considered as limiting the scope of the protection being sought.

1. Preparation of bottles:

All supports were sterilized by heating to 120 degrees Celsius (° C.) for 15 minutes (min). After drying for 24 hours at 37° C., they were dispensed into bottles using sterile technique. The amount introduced per bottle depended on the support being used and was adjusted to obtain a layer of material with a surface area approximately equivalent to the cross-sectional area of the bottle. The bottles with the supports inside were again heated to 120° C. for 15 min and then to each was added 40 ml of either a nutrient aerobic broth, VITAL AER (Ref. 52511 from bioMérieux) or a nutrient anaerobic broth, VITAL ANA (Ref. 52512 from bioMérieux) containing a marker or indicator for microbial growth as described and protected by the applicant's Patent EP-B-0.424.293.

The bottles were then placed in a vacuum and gassed in such a way as to generate an atmosphere suitable for the type of broth they contained (an aerobic atmosphere for VITAL AER and an anaerobic one for VITAL ANA). They were then stoppered and capped so that they are ready to use.

In order to make it possible to compare the effect of adding the supports dealt with in this invention to the culture media, bottles containing VITAL AER and ANA with no support were set up using exactly the same procedure except with respect to the preparation and addition of the support.

2. Testing of samples:

Using sterile technique, samples (both biological and non-biological) in which microorganisms were to be assayed were added to bottles which had been prepared as described above.

For simulations of positive blood cultures as might be encountered in the laboratory, the procedure was as follows. First, the test microorganisms were grown up on a suitable rich medium such as an agar medium, in most cases Columbia agar supplemented with 5% sheep blood.

For each strain, one or more colonies were picked to generate a suspension in distilled water. The density of this suspension was adjusted to 10⁸ cells/ml and diluted one million-fold. Using a sterile syringe, one milliliter of the diluted suspension was introduced into each VITAL bottle (with or without support) to which 5 ml of horse or human blood had been previously added. In this way, about one hundred microorganisms were added to each bottle.

The bottles containing the test sample were placed in the VITAL registered trademark system (ref.: 99105, 99106 or 99122 from bioMérieux) which acts as both incubator and reader. This system insures that the bottles are incubated at the correct temperature. Indicator kinetics were followed by optical measurements made through the bottles' clear glass walls. If the original sample contained microorganisms, these will proliferate during incubation and changes in the indicator contained in the reaction mixture will reflect the presence of the said microorganisms in the sample.

The efficiency of a given support was evaluated by comparing the detection times recorded using bottles with and without that support.

Examples according to the invention:

### Example 1

Detection of aerobic microorganisms

The assay was carried out in the following conditions:

VITAL AER bottles with and without support, prepared according to the procedure described in the paragraph entitled “Preparation of bottles”,

supports tested: polyethylene beads and cork fragments,

strains tested:

Haemophilus influenzae (bioMérieux ref.: JHI 8006064),

Kingella denitrificans (bioMérieux ref.: JKD 8311002), and

Staphylococcus saprophyticus (bioMérieux ref.: MSA 54677),

bacterial isolation medium: bioMérieux Columbia agar supplemented with 5% sheep blood,

simulations of positive blood cultures as indicated in the paragraph entitled “Testing of samples” with the addition, using sterile technique, of about one hundred microorganisms and 5 ml of horse blood per bottle,

determination of the time necessary to obtain a positive culture using the VITAL system (registered trademark), and

plotting of the growth curve for each bottle.

The results are shown in Table 1 which follows.

TABLE 1
Detection time in hours and time savings expressed as a
percentage of use of bottles with supports against
bottles without support for anaerobic or, optionally
aerobic, microorganisms

| | | Bottles | | Bottles | |
|---|---|---|---|---|---|
| | Bottles | containing | | containing | |
| | containing | polyethylene | | cork | |
| Strains | no support | beads | | fragments | |
| Haemophilus | 41.6 hours | 22.5 h | 45.9% | 31.2 h | 25% |
| influenzae | (h) | | | | |
| Kingella | 83 h | 52 h | 37.3% | 28 h | 66.3% |
| denitrificans | | | | | |
| Staphylococcus | 35.3 h | 27.5 h | 22.1% | 28.2 h | 20.1% |
| saprophyticus | | | | | |

Table 1 above shows that, in the presence of polyethylene beads, the detection time is reduced by a factor of between 22.1 and 45.9% (depending on the strain being tested) compared with bottles containing no support. In the presence of cork fragments, the range extends from 20.1 to 66.3%.

The effect of the supports can be seen in the growth curves presented in FIG. 1 for Haemophilus influenzae, FIG. 2 for Kingella denitrificans, and FIG. 3 for Staphylococcus saprophyticus.

### Example 2

Detection of obligate anaerobes

The assay was carried out in the following conditions:

VITAL ANA bottles with and without support, prepared according to the procedure described in the paragraph entitled “Preparation of bottles”,

supports tested: polyethylene beads and cork fragments,

strains tested:

Bacteroides fragilis (bioMérieux ref.: HBF 358),

Veillonella parvula (bioMérieux ref.: JKD 8311002), and

Clostridium sporogenes (bioMérieux ref.: ACO 100651),

bacterial isolation medium: bioMérieux Columbia agar supplemented with 5% sheep blood incubated in anaerobic conditions,

simulations of positive blood cultures as indicated in the paragraph entitled “Testing of samples” with the addition, using sterile technique, of about one hundred microorganisms and 5 ml of horse blood per bottle,

determination of the time necessary to obtain a positive culture using the VITAL system (registered trademark), and

plotting of the growth curve for each bottle.

The results obtained are shown in Table 2 which follows.

TABLE 2
Detection time in hours and time savings expressed as a
percentage of use of bottles with supports against
bottles without support for anaerobic microorganisms

| | | Bottles | | Bottles | |
|---|---|---|---|---|---|
| | Bottles | containing | | containing | |
| | containing | polyethylene | | cork | |
| Strains | no support | beads | | fragments | |
| Bacteroides | 86.5 h | 45 h | 48% | 62.2 h | 28.1% |
| fragilis | | | | | |
| Veillonella parvula | 176.4 h | 53.6 h | 69.6% | 64.5 h | 63.4% |
| Clostridium | 45.4 h | 27.7 h | 39% | 38 h | 16.3% |
| sporogenes | | | | | |

Table 2 above shows that the detection time compared with bottles containing no support is reduced by a factor of between 39 and 69.6% (depending on the strain being tested) in the presence of polyethylene beads, and by a factor of between 16.3 and 63.4% (depending on the strain being tested) in the presence of cork fragments.

The effect of the supports can be seen in the growth curves presented in FIG. 4 for Bacteroides fragilis, FIG. 5 for Veillonella parvula, and FIG. 6 for Clostridium sporogenes.

Whether Example 1 or Example 2 is under consideration, there is a clear reduction in detection time which reflects the faster rate at which cultures in bottles containing a support become positive. This is the result of faster modification of the indicator and applies to both aerobic and anaerobic microorganisms.

### Example 3

Further experiments on the detection of various aerobic, optionally anaerobic (AAF, anaérobie facultatif) , microorganisms

In this example, the FA strains tested included both bacteria and yeasts. Moreover, several different assays were carried out on each species tested. They belonged to the following different species or genera:

Neisseria,

Brucella,

Yeasts: Candida albicans, Candida parapsilosis, Candida guillermondii,

Staphylococcus,

Pseudomonas, and

Other strains belonging to: Haemophilis, Kingella, Eikenella and Micrococcus.

The results obtained are presented in Table 3 which follows. All the values shown correspond to the detection time in the presence of the relevant support expressed as a percentage of the detection time recorded in the bottles containing no support (taken as 100%).

TABLE 3
Effect of the nature of the support on the detection of
several aerobic microorganisms

| | Number of strains | | |
|---|---|---|---|
| Aerobic species | per species | Polyethylene | Polypropylene |
| Neisseria | 3 | 77.8 | 104.3 |
| Brucella | 3 | 80.4 | 124.0 |
| Yeasts | 6 | 70.6 | 103.6 |
| Staphylococcus | 5 | 83.6 | 95.9 |
| Pseudomonas | 2 | 74.6 | 174.3 |
| Other strains | 4 | 60.8 | 85.9 |
| Mean (total) for | 3.8 (23) | 74.6 | 114.7 |
| all strains | | | |

Table 3 clearly shows that the polyethylene support significantly reduced the detection times for all the species and genera tested, with times varying between 60.8and 83.6% of those recorded for the bottles which did not contain any support (100%).

The polypropylene support does not offer any benefit since it increased the detection time in a good number of cases, i.e. the result was over the 100% obtained with the reference bottles which did not contain any support. The result goes as high as 174.3% for Pseudomonas.

### Example 4

Investigation into the effect of the polyethylene support on the detection of anaerobic microorganisms

In this example, the strains used were anaerobic bacteria belonging to the following genera:

Bacteroides,

Clostridium,

Peptostreptococcus,

Veillonella, and

Fusobacterium.

The results obtained are presented in FIG. 4 which follows.

All the results are shown as percentages which, as in Table 3, correspond to the detection time in the presence of the support expressed as a percentage of the detection time recorded in the bottles which contained no support (taken as 100%).

TABLEAU 4
Effect of the support on the detection of
severalanaerobic microorganisms

| Anaerobic | Number of strains per | |
|---|---|---|
| species | species | Polyethylene |
| Clostridium | 2 | 69.0 |
| Bacteroides | 7 | 64.5 |
| Peptostreptococcus | 2 | 97.3 |
| Veillonella | 2 | 27.6 |
| Fusobacterium | 1 | 70.6 |
| Mean (total) | 2.8 (14) | 65.8 |
| for all strains | | |

Table 4 above shows reductions in detection time (compared with the bottles containing no support) of between 2.7and 72.4%, depending which anaerobic species was being tested.

Thus, as the examples above show, the efficiency of the method is proven. This is true for the method used for blood cultures (in which blood is assayed for the presence of microorganisms) and also for other applications based on the culture of microorganisms.

This method essentially consists of four or five sequential steps.

First, the sample to be tested is added into the receptacle containing a culture medium. Second, it is necessary to further add into the said receptacle at least one sterile, inert, solid support. Third (if necessary), the receptacle may be sealed. Fourth, the culture is incubated at a suitable temperature. Fifth and finally, the variation in some characteristic related to the growth and/or metabolism of the microorganism to be detected is monitored.

This characteristic can in general be of two different kinds.

One kind corresponds to a chemical indicator, e.g. a colored, fluorescent, or chromogenic agent which is added to the receptacle before incubation. The other kind consists of some physicochemical or electrical parameter which is modified by the simple growth of the microorganisms to be detected. In the latter case, it is unnecessary to add any substance in order to be able to monitor variation in the parameter. The monitoring may be automated or may be done manually, i.e. by eye. In the case of automated monitoring, the parameter(s) can be monitored by sensors which, for example may measure pressure, CO₂ production, turbidity, oxidation/reduction potential or pH. This list is not limiting and any physicochemical or electrical characteristic which might vary according to a microorganism's metabolic activity is covered.

With respect to the suitable temperature mentioned in the description of the method given above, this will depend on the family, species or genus of the microorganism being tested. In practice, this varies enormously and can range between 0 and 100° C. for different microorganisms. The correct temperatures are known to those skilled in the art and/or are easy to determine.

As well as being suitable for the testing of samples in which it is desired to detect the presence of microorganisms, this method can also be used in sterility testing (of biological fluids such as blood, cerebrospinal fluid, pleural fluid, urine, etc.; and non-biological samples such as water, food products or pharmaceutical products).

The results presented in the examples given above demonstrate the impact of growth supports on the detection times for both aerobic and anaerobic microorganisms (with anaerobic organisms requiring and O₂-free N₂/CO₂ atmosphere) . The detection time was reduced to a significant extent in most cases.

The examples given are limited to the use of certain supports and to the investigation of their effect on the detection of certain microorganisms. However, similar results have already been obtained with other supports and in investigations extended to cover other microorganisms. Therefore, the scope of this invention is not limited to the species and supports studied here.

In conclusion, the presence of a support according to the invention significantly reduces the time it takes to detect the presence of microorganisms in a sample, whatever their metabolism, be it aerobic or anaerobic. Such a result was obtained without having to investigate the effect of enhanced oxygenation or improved exposure of the microorganisms to the oxygen which might be present in the medium.

1. A method for detecting the presence in a sample, contained in a sterile receptacle, of at least one anaerobic microorganism, the sample being in contact with a culture medium, which consists in:
adding into the receptacle at least one sterile, inert, solid support,
incubating at a suitable temperature, and
observing the variation in at least one characteristic related to the presence of the microorganism(s) to be detected in said receptacle.

2. A method for detecting the presence in a sample, contained in a sterile receptacle, of at least one aerobic microorganism, the sample being in contact with a culture medium, which consists in:
adding into the receptacle at least one sterile, inert, solid support in such a quantity as to obtain a layer of material with a surface area approximately equivalent to that of the interface between the sample and the gaseous atmosphere in the receptacle,
incubating at a suitable temperature, and
monitoring the variation in at least one characteristic related to the presence of the microorganism(s) to be detected in said receptacle.

3. A method, according to either of claims 1 or 2, characterized in that the characteristic monitored is due to variation in at least one chemical indicator added into the receptacle before incubation, e.g. a colored or fluorescent indicator, and/or at least one physicochemical or electrical parameter, e.g. CO₂ production, pressure, turbidity, oxidation/reduction potential and/or pH.

4. A method, according to any of claims 1 through 3, characterized in that the sample is biological in nature, e.g. blood, cerebrospinal fluid, pleural fluid or urine, or non-biological, e.g. water, food products, or pharmaceutical products.

5. A method, according to any of claims 1 through 4, characterized in that variations in the indicator(s) are detected optically through all or part of at least one of the walls (which are transparent) of the receptacle, and/or changes in the parameter(s) are detected by means of physicochemical or electrical sensors.

6. A method, according to any of claims 1 through 5, characterized in that it is applied to anaerobic microorganisms.

7. A Method, according to any of claims 1 through 6, characterized in that it is used in a sterility test.

8. A method according to any of claims 1 through 7, characterized in that the sterile, inert, solid support is made of natural materials, e.g.:
silica beads,
small glass beads (solid, hollow or porous),
quartz particles,
grains of sand,
vermiculite, zeolite and/or feldspar particles,
glass wool and/or rock wool,
clay beads, and
cork fragments.

9. A method according to any of claims 1 through 7, characterized in that the sterile, inert, solid support is made of artificial materials, e.g.:
polystyrene beads,
polyethylene beads,
polypropylene beads,
clusters of small polyethylene beads, with variable pore-size and dimensions,
growth supports in the form of small beads used in tissue culture,
latex beads,
gelatin-coated beads, and
resin beads.

10. A method according to any of claims 1 through 7, characterized in that the sterile, inert, solid support consists of an element of any shape made of polyethylene.

11. A method, according to either of claims 1 or 2, characterized in that the support consists of beads or particles with a diameter of between 1 μm and 10 mm especially between 0.1 mm and 5 mm.
